(19) United States
(12) Patent Application Publication
(10) Pub. No.: US 2024/0053344 A1
(43) Pub. Date: Feb. 15, 2024

(54) METHODS FOR DETECTING SQUAMOUS CANCER

(71) Applicant: The Trustees of Columbia University in the City of New York, New York, NY (US)

(72) Inventors: Fatemeh Momen-Heravi, Teaneck, NJ (US); Akiva Mintz, Paramus, NJ (US); Gordana Vunjak-Novakovic, New York, NY (US); Keith Yeager, Springfield, NJ (US)

(21) Appl. No.: 18/381,868

(22) Filed: Oct. 19, 2023

Related U.S. Application Data
(63) Continuation of application No. PCT/US2022/026073, filed on Apr. 23, 2022.
(60) Provisional application No. 63/178,727, filed on Apr. 23, 2021.

(51) Int. Cl. G01N 33/574; G01N 33/68; G01N 1/40

(52) U.S. Cl. CPC G01N 33/57488; G01N 33/68; G01N 1/40; G01N 2800/52

(57) ABSTRACT

A method of detecting or diagnosing squamous cancers in a sample from a subject, including isolating extracellular vesicles from the sample, wherein the extracellular vesicles are cancer specific.

### CROSS-REFERENCE TO RELATED APPLICATIONS

This application claims priority to U.S. Provisional Application No. 63/178,727 filed Apr. 23, 2021, entitled “Extracellular Vesicles-Associated Proteins as Biomarkers for Head and Neck Cancer,” which is incorporated by reference in its entirety herein.

### FIELD

The disclosed subject matter describes methods of detecting or diagnosing squamous cancers in a sample from a subject, including isolating exosomes and/or extracellular vesicles from the sample.

### BACKGROUND

Head and neck squamous cell carcinoma (HNSCC) is one of the most prevalent malignancies in the world, with approximately 53,000 cases diagnosed annually. Risk factors such as tobacco use, alcohol use, and human papilloma virus (HPV) are associated with an increased risk of developing HNSCC. While improvements in treatment and therapy have led to an increase in the survival rate of HNSCC patients, when diagnosed, 60% of patients have advanced stage disease, often stage III or IV, which is associated with unfavorable prognosis. Also, recurrence of HNSCC within 2-3 years is a common phenomenon. Therefore, improving early detection and understanding the biology of HNSCC tumor progression are necessary for better treatment.

Extracellular vesicles (EVs), including exosomes and microvesicles, are emerging as a popular means for early detection of malignant tumors due to their important role in intracellular signaling and cellular homeostasis. EVs are membrane-bound vesicles with a phospholipid bilayer that can horizontally transfer molecules or activate receptors on target cells. The contents of these vesicles include membrane proteins, cytoskeleton components, DNAs, and RNAs. They are involved in development, growth, and cell to cell communication. Different cells produce unique EVs and associated contents based on the cell's characteristics and microenvironment. Recently, these vesicles have been used as non-invasive biomarkers due to their presence in biofluids such as saliva, blood, urine, and cerebral spinal fluid. An advantage of EVs is the high conservation and enrichment of biological information from their parental source. In disease states or when exposed to stress, EVs adapt and alter contents based on the tumor microenvironment (TME) to contain different cargo for signaling, reflecting the pathological state of their origin. These EVs protect survival of cancer cells from the immune system by creating an immunosuppressive environment.

EVs act as one of the important mediators of TME and tumor paracrine signaling. It has been demonstrated that tumor-derived EVs suppress anticancer responses through promoting CD8+ T-cell apoptosis. Tumor EVs prevent their own breakdown through promoting formation of immunosuppressive tumor associated macrophages. Not only do tumor EVs escape immune response and induce immunosuppressive profile, they also activate tumor growth and metastasis. Their contents can include oncogenic epidermal growth factor receptors and can horizontally transfer activated oncogenes between neoplastic cells. Tumor EVs also activate the epithelial-mesenchymal transition (EMT) pathway through promoting loss of epithelial-cell adhesion, alteration of the tumors structure, and release of cancer cells to metastasis.

The microRNA content of EVs has been extensively studied for its role as a potential biomarker due to its involvement in cell-cell communication, regulation of TME, and association in pathological processes such as tumorigenesis, metastasis, and progression. Recently, proteomic analysis has gained more attention in an attempt to understand tumor molecular effectors and metastasis and develop diseases-associated biomarkers. Proteins have become main targets in therapy due to the dysregulation of secreted proteins by TME. Proteomic cargo of EVs is dependent on the cell of origin. While many studies attempted to identify protein biomarkers for HNSCC in tumor tissue, there is little proteomic data of EVs in patients with HNSCC versus patients free of disease. Also, the functional role of EV-associated proteins in HNSCC is not known.

There is a need for method and a tool for detecting or diagnosing cancers in a subject using extracellular vesicles

### SUMMARY

The present disclosure provides a method of identifying the presence or absence of squamous cancer in a subject comprising: determining the expression level of at least one protein selected from ITGB3, ITGA2B, H2B1K, IGHE, SPTA1, MYH9, EF1A1, MEPE, DHX38, and combinations thereof in a biological sample from the subject.

The present disclosure provides a method of identifying the presence or absence of squamous cancer in a subject comprising: determining the expression level of at least one protein selected from ITGB3, ITGA2B, H2B1K, IGHE, SPTA1, MYH9, EF1A1, MEPE, DHX38, and combinations thereof in a biological sample from the subject; determining at least one reference value in the biological sample; normalizing the expression level of the at least one protein to the at least one reference value to obtain a normalized expression level of the at least one protein; comparing the normalized expression level of the at least one protein to a predetermined cutoff value; and identifying the presence of squamous cancer in the subject when the normalized expression level of the at least one protein is greater than the predetermined cutoff value or identifying the absence of squamous cancer in the subject when the normalized expression level of the at least one protein is less than or equal to the predetermined cutoff value.

The present disclosure provides a method of identifying the presence or absence of squamous cancer in a subject comprising: determining the expression level of at least one protein selected from ITGB3, ITGA2B, H2B1K, IGHE, SPTA1, MYH9, EF1A1, MEPE, DHX38, and combinations thereof in a biological sample from the subject; determining at least one reference value in the biological sample; normalizing the expression level of the at least one protein to the at least one reference value to obtain a normalized expression level of the at least one protein; comparing the normalized expression level of the at least one protein to a predetermined cutoff value; and wherein the biological sample comprises at least one extracellular vesicle (EV) nucleic acid extracted from at least one EV; identifying the presence of squamous cancer in the subject when the normalized expression level of the at least one protein is greater than the predetermined cutoff value or identifying the absence of squamous cancer in the subject when the normalized expression level of the at least one protein is less than or equal to the predetermined cutoff value.

The present disclosure provides a method of identifying the presence or absence of squamous cancer in a subject comprising: determining the expression level of at least one gene selected from A1BG, SERPINC1, APOA4, HBB, AFM, TLN1, F5, SERPINA5, AZGP1, SERPINB3, and ALB in a biological sample from the subject, wherein the biological sample comprises at least one extracellular vesicle (EV) nucleic acid extracted from at least one EV; determining at least one reference value in the biological sample; normalizing the expression level of the at least one gene to the at least one reference value to obtain a normalized expression level of the at least one protein; comparing the normalized expression level of the at least one gene to a predetermined cutoff value; and identifying the presence of squamous cancer in the subject when the normalized expression level of the at least one gene is greater than the predetermined cutoff value or identifying the absence of squamous cancer in the subject when the normalized expression level of the at least one gene is less than or equal to the predetermined cutoff value.

The squamous cancer is head and neck cancer.

In methods of the present disclosure, the at least one EV nucleic acid comprises EV DNA, EV RNA or a combination of EV DNA and EV RNA. At least one EV nucleic acid comprises EV RNA.

At least one EV can be isolated from a bodily fluid sample from the subject by a method comprising filtration, size exclusion chromatography, ion exchange chromatography, density gradient centrifugation, centrifugation, differential centrifugation, immunoabsorbent capture, affinity purification, affinity enrichment, affinity exclusion microfluidic separation, ultracentrifugation, nanomembrane ultrafiltration or any combination thereof. The bodily fluid sample is filtered or pre-processed to remove cells, cellular debris or any combination thereof prior to the isolation of at least one EV.

In methods of the present disclosure, a predetermined cutoff value can have a sensitivity of at least 50%, or at least 60%, or at least 70%, or at least 80%, or at least 90%, or at least 95%, or at least 99%.

In methods of the present disclosure, a predetermined cutoff value can have a sensitivity of at least 50%, or at least 60%, or at least 70%, or at least 80%, or at least 90%, or at least 95%, or at least 99%.

A subject can have previously had squamous cancer. A subject can be suspected of having squamous cancer.

The methods of the present disclosure can further comprise administering to a subject having squamous cancer at least one therapeutically effective amount of at least one therapy. A therapy can comprise surgery, radiation therapy, chemotherapy, intravesical therapy, anti-cancer therapy, immunotherapy, intravesical immunotherapy, targeted-drug therapy, intravesical chemotherapy, or any combination thereof. Surgery can comprise resection of the tumor.

The methods of the present disclosure can further comprise providing a treatment recommendation for the subject. The treatment recommendation can comprise recommending further diagnostic tests, recommending the administration of at least one therapy or any combination thereof.

The methods of the present disclosure can further comprise providing an indication of a resistance or response to the therapy

A method of detecting or diagnosing squamous cancers in a sample from a subject, including isolating extracellular vesicles from the sample, wherein the extracellular vesicles are cancer specific. In some embodiments, the cancer specific extracellular vesicles have a mean size of 106 nm.

In some embodiments, the cancer specific extracellular vesicles express protein cell surface markers chosen from the group consisting of integrin proteins ITGB3 and ITGA2B. In some embodiments, the method further includes detecting and/or measuring the level of at least one protein present in the cancer specific extracellular vesicles. In some embodiments, at least one protein is chosen from the group consisting of ITGB3, ITGA2B, H2B1K, IGHE, SPTA1, MYH9, EF1A1, MEPE, DHX38, and combinations thereof.

In some embodiments, the method further includes detecting and/or measuring the level of at least one miRNA present in the cancer specific extracellular vesicles. In some embodiments, the at least one miRNA is chosen from the group consisting of miR-34a, miR-183, miR-21, and combinations thereof.

In some embodiments, the method further includes detecting and/or measuring the level encapsulated RNA, wherein the RNA encodes cancer specific proteins. In some embodiments, the squamous cancer is head and neck cancer. In some embodiments, the sample is liquid.

A platform for the isolation of cancer specific exosomes and/or extracellular vesicles in a sample is provided, including aptamers which are specific for protein cell surface markers of the cancer specific exosomes and/or extracellular vesicles. In some embodiments, the platform includes a chip having reagents for RNA isolation from the cancer specific exosomes and/or extracellular vesicles. In some embodiments, the protein cell surface markers are chosen from the group consisting of integrin proteins ITGB3 and ITGA2B. In some embodiments, the aptamers specific for at least one protein chosen from the group consisting of ITGB3, ITGA2B, H2B1K, IGHE, SPTA1, MYH9, EF1A1, MEPE, DHX38, and combinations thereof.

In some embodiments, the platform detects and/or measures the level of at least one miRNA present in the cancer specific exosomes and/or extracellular vesicles chosen from the group consisting of miR-34a, miR-183, miR-21, and combinations thereof.

A platform for the isolation of cancer specific extracellular vesicles in a sample is provided including isolating encapsulated RNA, wherein the RNA encodes cancer specific proteins. In some embodiments, the cancer specific proteins are chosen from the group consisting of ITGB3, ITGA2B, H2B1K, IGHE, SPTA1, MYH9, EF1A1, MEPE, DHX38, and combinations thereof. In some embodiments, the platform further includes detecting and/or measuring the level of at least one miRNA present in the cancer specific extracellular vesicles chosen from the group consisting of miR-34a, miR-183, miR-21, and combinations thereof. In some embodiments, the platform includes aptamers which are specific for protein cell surface markers of the cancer specific exosomes and/or extracellular vesicles.

In some embodiments, the protein cell surface markers are chosen from the group consisting of integrin proteins ITGB3 and ITGA2B. In some embodiments, the squamous cancer is head and neck cancer.

Any of the above aspects can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 is a schematic view of a platform for isolation of cancer-specific exosomes/extracellular vesicles and RNA isolation for qPCR base biomarker analysis assay.

FIG. 2A is a Western blot analysis of EV markers (CD63, CD81) and cellular marker GRP04 in HNSCC control EVs, tumor EVs, and FADU (a HNSCC cell line) cell lysate.

FIG. 2B is a nanoparticle tracking analysis of EV size and concentration (upper line: HNSCC EVs, lower line: healthy EVs).

FIG. 2C is a volcano plot of differentially expressed proteins in HNSCC EVs versus control EVs (n=10 control; n=14 HNSCC). Data is presented as −log 10 p value and Log2 Fold change. Significantly differentially expressed proteins are depicted above the horizontal line.

FIGS. 2D-E are pathway enrichment analysis (FIG. 1D Reactome and FIG. 1E GO biological process) of proteomic signature of EV s. * indicates p<0.05.

FIG. 3A is a pathway analysis and gene ontology analysis (GO biological process, GO molecular function, KEGG, REACTOME) of proteomic signature of EVs isolated in plasma of patients with HNSCC.

FIG. 3B is a Venn diagram of EV proteins isolated from plasma of HNSCC patients versus EV s isolated from plasma of healthy individuals.

FIG. 3C illustrates the level of ITGB3 was quantified in HNSCC EVs (n=22) and healthy EV s (n=20) by ELISA. Mann Whitney U test was used for statistical analysis.

FIG. 3D illustrates Receiver Operating Characteristic (ROC) curve of EV ITGB3 as biomarker to predict HNSSC. The curve was constructed using data from EV ITGB3s levels of HNSCC patients (n=22) and age- and sex-matched healthy individuals (n=20). The filled line represents the area under the curve (AUC).

FIG. 3E is a Venn diagram of EV proteins isolated for plasma of HNSCC patients versus EV proteins isolated from HPV (−) HNSCC cell lines.

FIG. 3F is a KEGG pathway analysis of 92 common proteins in EV s isolated in plasma of patients with HNSCC and HPV (−) HNSCC cell line. * indicates p<0.05.

FIGS. 4A-E illustrate EVs isolated from plasma of 29 HNSCC patients and 10 healthy controls by LEGENDplex™ Human Fibrinolysis Panel (FIG. 4A Antithrombin; FIG. 4B Factor XIII; FIG. 4C Fibrinogen; FIG. 4D Plasminogen; and FIG. 4E Prothrombin). Data is presented in ng/ml.

FIG. 4F illustrates clotting time quantified in the whole blood after coculture of HNSCC EVs or control EVs (25 ng/ml-50 ng/ml).

FIG. 4G illustrates HNSCC tumor EVs and control EVs (25 ng/ml-50 ng/ml) incubated in triplicates with platelet rich plasma for 2h at 37° C. Percentage of CD62P+ platelets were identified by flow cytometry.

FIG. 4H illustrates HNSCC EVs or control EVs (25 ng/ml-50 ng/ml) cocultured with freshly drawn whole blood (500 ul). The blood was incubated for 2h at 37° C. and the blood was aspirated and centrifuged to obtain platelet poor plasma and analyzed by a thrombin-antithrombin complex (TAT) ELISA assay. For FIGS. 3A-H, * indicates p<0.05; ** indicates p<0.01; *** indicates p<0.001. **** indicates p<0.0001. Mann Whitney U test was used for statistical analysis. Data is presented as mean±SEM. Experimental data is result of at least three independent experiments.

FIG. 5A illustrates genomic alterations (gain, amplification, mRNA high) of proteins which were highly abundant in EVs were explored in TCGA HNSCC data (n=530).

FIGS. 5B-C illustrate GSEA analysis of tumors which demonstrated high abundance of dysregulated proteins in HNSCC EV s versus tumors with low abundance of dysregulated proteins in HNSCC. FIG. 5B illustrates Epithelial-mesenchymal transition and genes with highest enrichment score. FIG. 5C illustrates Interferon Gamma response and genes with highest enrichment score.

FIG. 5D-E illustrate high abundance of proteins in HNSCC EVs associated with significantly higher fraction of genome altered (FIG. 5D) and higher mutation count (FIG. 5E) in TCGA data (N=530). (Kruskal Wallis test, adjusted for multiple comparison by false discovery rate of <0.05) ** indicates p<0.01; * indicates p<0.05.

FIGS. 6A-B illustrate Kaplan-Meier survival curves of patients with high levels of AZGPl (FIG. 6A) and SERPINA5 (FIG. 6B).

FIG. 6C illustrates percentage of HPV+ in patients with high levels of AZGPl and low levels of AZGPl (AZGPl high, n=166; AZGPl unaltered or low, n=330).

FIG. 6D illustrates the association of AZGPl expression with tumor immune-infiltrate in HNSCC cohort of TCGA data. Data is presented as partial Spearman's rho of AZGPl expression level (log 2 TPM) and infiltration. ** indicates p<0.01

FIG. 7A is a bar chart showing genomic alterations of EV-enriched proteins in other solid tumors such as bladder and lung cancer.

FIGS. 7B-E illustrate Kaplan-Meier survival curves for Overall survival (FIG. 7B), the disease-free survival (FIG. 7C), disease-specific survival (FIG. 7D), and progression-free survival (FIG. 7E).

FIG. 8A is a dot plot showing expression for miR-183.

FIG. 8B is a dot plot showing expression for miR-21.

### DETAILED DESCRIPTIONS OF EXEMPLARY EMBODIMENTS

The present disclosure provides methods for providing a clinical assessment of a subject in need therefore. The clinical assessment can include, but is not limited to, diagnosing a subject, monitoring a subject, recommending a treatment for a subject or prognosing a subject. In some aspects, the clinical assessment is informed by the analysis of the contents of extracellular vesicles (EVs).

Reference has been made in detail to select embodiments of the disclosed subject matter, examples of which are illustrated in the accompanying drawings.

The present disclosure provides a method of identifying the presence or absence of squamous cancer in a subject comprising: determining the expression level of at least one protein selected from ITGB3, ITGA2B, H2B1K, IGHE, SPTA1, MYH9, EF1A1, MEPE, DHX38, and combinations thereof in a biological sample from the subject.

The present disclosure provides a method of identifying the presence or absence of squamous cancer in a subject comprising: determining the expression level of at least one protein selected from ITGB3, ITGA2B, H2B1K, IGHE, SPTA1, MYH9, EF1A1, MEPE, DHX38, and combinations thereof in a biological sample from the subject; determining at least one reference value in the biological sample; normalizing the expression level of the at least one protein to the at least one reference value to obtain a normalized expression level of the at least one protein; comparing the normalized expression level of the at least one protein to a predetermined cutoff value; and identifying the presence of squamous cancer in the subject when the normalized expression level of the at least one protein is greater than the predetermined cutoff value or identifying the absence of squamous cancer in the subject when the normalized expression level of the at least one protein is less than or equal to the predetermined cutoff value.

The present disclosure provides a method of identifying the presence or absence of squamous cancer in a subject comprising: determining the expression level of at least one protein selected from ITGB3, ITGA2B, H2B1K, IGHE, SPTA1, MYH9, EF1A1, MEPE, DHX38, and combinations thereof in a biological sample from the subject; determining at least one reference value in the biological sample; normalizing the expression level of the at least one protein to the at least one reference value to obtain a normalized expression level of the at least one protein; comparing the normalized expression level of the at least one protein to a predetermined cutoff value; and wherein the biological sample comprises at least one extracellular vesicle (EV) nucleic acid extracted from at least one EV; identifying the presence of squamous cancer in the subject when the normalized expression level of the at least one protein is greater than the predetermined cutoff value or identifying the absence of squamous cancer in the subject when the normalized expression level of the at least one protein is less than or equal to the predetermined cutoff value.

The present disclosure provides a method of identifying the presence or absence of squamous cancer in a subject comprising: determining the expression level of at least one gene selected from A1BG, SERPINC1, APOA4, HBB, AFM, TLN1, F5, SERPINA5, AZGP1, SERPINB3, and ALB in a biological sample from the subject, wherein the biological sample comprises at least one extracellular vesicle (EV) nucleic acid extracted from at least one EV; determining at least one reference value in the biological sample; normalizing the expression level of the at least one gene to the at least one reference value to obtain a normalized expression level of the at least one protein; comparing the normalized expression level of the at least one gene to a predetermined cutoff value; and identifying the presence of squamous cancer in the subject when the normalized expression level of the at least one gene is greater than the predetermined cutoff value or identifying the absence of squamous cancer in the subject when the normalized expression level of the at least one gene is less than or equal to the predetermined cutoff value.

The squamous cancer can be head and neck cancer.

In some aspects, the at least one EV nucleic acid can comprise EV DNA, EV RNA or a combination of EV DNA and EV RNA. At least one EV nucleic acid comprises EV RNA.

In some aspects, at least one EV can be isolated from a bodily fluid sample from the subject by a method comprising filtration, size exclusion chromatography, ion exchange chromatography, density gradient centrifugation, centrifugation, differential centrifugation, immunoabsorbent capture, affinity purification, affinity enrichment, affinity exclusion microfluidic separation, ultracentrifugation, nanomembrane ultrafiltration or any combination thereof. The bodily fluid sample is filtered or pre-processed to remove cells, cellular debris or any combination thereof prior to the isolation of at least one EV.

In methods of the present disclosure, a predetermined cutoff value can have a sensitivity of at least 50%, or at least 60%, or at least 70%, or at least 80%, or at least 90%, or at least 95%, or at least 99%.

In methods of the present disclosure, a predetermined cutoff value can have a sensitivity of at least 50%, or at least 60%, or at least 70%, or at least 80%, or at least 90%, or at least 95%, or at least 99%.

A subject can have previously had squamous cancer. A subject can be suspected of having squamous cancer.

The methods of the present disclosure can further comprise administering to a subject having squamous cancer at least one therapeutically effective amount of at least one therapy. A therapy can comprise surgery, radiation therapy, chemotherapy, intravesical therapy, anti-cancer therapy, immunotherapy, intravesical immunotherapy, targeted-drug therapy, intravesical chemotherapy, or any combination thereof. Surgery can comprise resection of the tumor.

The methods of the present disclosure can further comprise providing a treatment recommendation for the subject. The treatment recommendation can comprise recommending further diagnostic tests, recommending the administration of at least one therapy or any combination thereof.

The methods of the present disclosure can further comprise providing an indication of a resistance or response to the therapy

Based on the hypothesis that the protein cargos of EVs could be indicative of HNSCC prognosis and mechanistic means of modulating TME and tumor systemic effects, the proteomic cargo of EVs isolated from plasma of HNSCC patients has been characterized herein. It was found herein that certain proteins were upregulated or expressed inclusively in HNSCC EVs and HNSCC cell lines. Many of those proteins belonged to hemostasis and coagulation pathways. Functional analysis identified role of EVs in promoting blood coagulation pathways. Bioinformatic analysis in the cancer genome atlas (TCGA) confirmed genomic alteration in form of gain, amplification, and high mRNA levels in EVs and enriched coagulation and hemostasis related genes in HNSCC and other solid tumors. The presence of those genomic alterations and high expression of those genes were associated with lower survival in HNSCC.

EVs are important mediators of tumor microenvironments and systemic effects of cancer. EV-associated protein biomarkers and the functional role of EVs in HNSCC was identified. A key novel finding was that Integrin beta-3 (ITB3) and Integrin alpha-IIb (ITGA2B) were found to be present exclusively in plasma of HNSCC-derived EVs and not in healthy subjects. Many enriched proteins in HNSCC-derived EVs which belong to hemostasis and coagulation pathways and the mechanistic studies performed herein showed the role of HNSCC EVs in promoting blood coagulation and platelet activation. Bioinformatic analysis in the cancer genome atlas confirmed high frequency of genomic alteration in the form of gain, amplification, and high mRNA levels in hemostasis-related genes in HNSCC tumors. Enrichment of these genes in HNSCC and other solid tumors was prevalent and associated with high tumor mutational burden, epithelial-mesenchymal transition, and suppression of interferon gamma signaling. High levels of AZGP1, an abundant protein in HNSCC EVs, was associated with immunologically cold tumors and poor survival outcomes. These data indicate the functional role of tumor EVs in coagulation and their diagnostic utility as disease associated-biomarkers.

Exemplary methods and platforms are disclosed herein.

The present disclosure provides a method of detecting or diagnosing squamous cancers in a sample from a subject, comprising isolating extracellular vesicles from the sample, wherein the extracellular vesicles are cancer specific. The squamous cancer may include head and neck cancer. The cancer specific extracellular vesicles have a mean size of 106 nm. The cancer specific extracellular vesicles express protein cell surface markers chosen from the group consisting of integrin proteins ITGB3 and ITGA2B. The method may further include detecting and/or measuring the level of at least one protein present in the cancer specific extracellular vesicles, the at least one protein selected from the group consisting of ITGB3, ITGA2B, H2B1K, IGHE, SPTA1, MYH9, EF1A1, MEPE, DHX38, and combinations thereof. The method may further includes detecting and/or measuring the level of at least one miRNA present in the cancer specific extracellular vesicles, the at least one miRNA selected from the group consisting of miR-34a, miR-183, miR-21, and combinations thereof. The method may further includes detecting and/or measuring the level encapsulated RNA, wherein the RNA encodes cancer specific proteins.

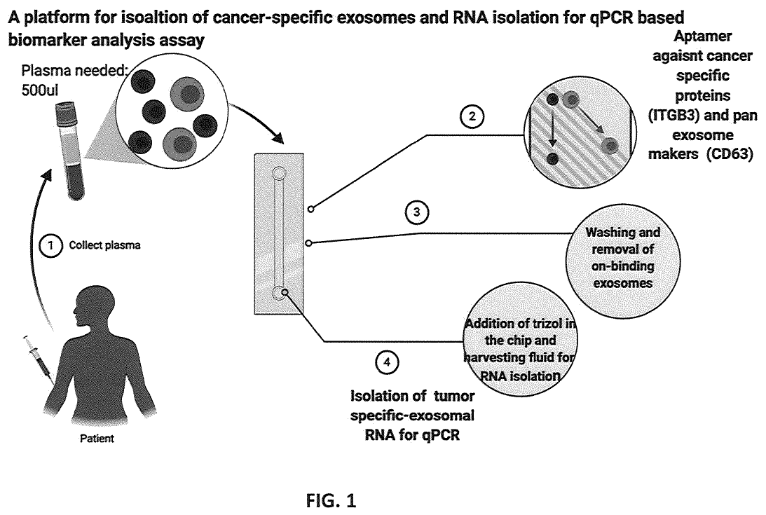

The present disclosure provides a platform for the isolation of cancer specific extracellular vesicles in a sample, comprising aptamers which are specific for protein cell surface markers of the cancer specific exosomes and/or extracellular vesicles. (FIG. 1) The platform comprises a chip comprising reagents for RNA isolation from the cancer specific exosomes and/or extracellular vesicles. The protein cell surface markers are chosen from the group consisting of integrin proteins ITGB3 and ITGA2B. The aptamers specific for at least one protein are selected from the group consisting of ITGB3, ITGA2B, H2B1K, IGHE, SPTA1, MYH9, EF1A1, MEPE, DHX38, and combinations thereof. The platform detects and/or measures the level of at least one miRNA present in the cancer specific extracellular vesicles chosen from the group consisting of miR-34a, miR-183, miR-21, and combinations thereof.

The present disclosure provides a platform for the isolation of cancer specific extracellular vesicles in a sample including isolating encapsulated RNA, wherein the RNA encodes cancer specific proteins. In some embodiments, the cancer specific proteins are chosen from the group consisting of ITGB3, ITGA2B, H2B1K, IGHE, SPTA1, MYH9, EF1A1, MEPE, DHX38, and combinations thereof. In some embodiments, the platform further includes detecting and/or measuring the level of at least one miRNA present in the cancer specific extracellular vesicles chosen from the group consisting of miR-34a, miR-183, miR-21, and combinations thereof. In some embodiments, the platform includes aptamers which are specific for protein cell surface markers of the cancer specific exosomes and/or extracellular vesicles. In some embodiments, the protein cell surface markers are chosen from the group consisting of integrin proteins ITGB3 and ITGA2B.

### EXAMPLES

TCGA samples. The analysis included the TCGA pan-cancer atlas dataset, which includes 10,522 tumors across 33 cancer types including 528 HNSCC samples. The following data is available at gdc.cancer.gov/node/977: cancer type, HPV status, mutation status, copy number variation, and mRNA gene expression. Pearson's correlation coefficient, and correlation coefficients with a false discovery rate (FDR)-adjusted p value of <0.05 were considered statistically significant. The Mann-Whitney U test was used to compare different groups of samples. Survival analysis was performed on TCGA data by constructing Cox proportional-hazards model and Kaplan Meier as implemented in R. Estimation of individual immune subtype fractions was performed by TIMER.

Gene Set Enrichment Analysis (GSEA). First, within the HNSCC samples of the TCGA, RNA-sequencing analysis was performed using DESEQ2 to identify the differentially expressed genes using R. Genes that were differently expressed between each condition with a FDR-adjusted p-value of <0.1 were analyzed by the GSEA pre-ranked algorithm to look for enrichment of these genes in the GSEA hallmark datasets.

Clinical samples. Clinical samples and plasma were obtained from the tumor bank of Columbia University Irving Medical Center, Biomarker Core of Herbert Irving Comprehensive Cancer Center or University of Massachusetts Medical School Conquering Diseases Biorepository. All samples were collected based on an institutional review board guideline. Plasma and tissue samples were obtained from 48 histologically-confirmed HNSCC patients and 48 age- and sex-matched healthy controls. None of the patients received chemotherapy or radiotherapy before sample collection. All cases were HPV negative and from oral cavity. The mean (SD) of the age of HNSCC was 65 (6.4) years with the female to make ratio of 7/3, and the mean (SD) age of healthy controls was 64 (7.0) with the male to female ratio of 8/2.

EV isolation. EVs isolation was carried out as described to collect the small EV fraction (exosomes). Briefly, 250 μL plasma was centrifuged at 1,500 g for 5 minutes (4° C.) to remove cells followed by 10,000 g for 20 minutes (4° C.) to remove cellular debris. The plasma was then filtered through a 0.2 μm syringe filter (Millipore, Billerica, MA, USA) to exclude vesicles >200 nm, and EVs were precipitated with Exoquick-TC™ according to the manufacturer's guidelines. After precipitation, the EVs pellet was washed three times to remove any contaminant and resuspended in PBS in 10 μL aliquots and stored at −80° C.

Nanoparticle Tracking Analysis (NTA). The size and concentration of EVs in plasma were identified by a NanoSight NS300 system (NanoSight, Amesbury, UK) equipped with a fast video capture and Nanoparticle Tracking Analysis (NTA) software. The instrument was calibrated with 100 nm polystyrene beads (Thermo Scientific, Fremont, CA, USA) before each measurement. The samples were captured for 90 s at room temperature. NTA software was used to identify the concentration of the particles (particles/ml) and size distribution (in nanometer). Each sample was measured at least four times.

Proteomic, protein quantification, and pathway enrichment analysis. For sample preparation, proteins were excised from polyacrylamide gels via SDS-PAGE and Coomassie blue dye staining. Protein specimens were reduced and alkylated using DTT and iodoacetamide, followed by an over overnight digestion using trypsin at 37° C. The peptides were de-salted using the solid phase extraction method. MS/MS spectra was generated by a Thermo Scientific Orbitrap Fusion Lumos. Scaffold Q+ (version 4.8.7, Proteome Software Inc., Portland, OR) was used to quantitate Label Free Quantitation peptide and protein identifications. Peptide identifications were accepted if they could be established at greater than 80.0% probability by the Peptide Prophet algorithm (22), with Scaffold delta-mass correction. Protein identifications were accepted if they could be established at greater than 80.0% probability and contained at least 2 identified peptides. Protein probabilities were assigned by the Protein Prophet algorithm. Proteins that contained similar peptides and could not be differentiated based on MS/MS analysis alone were grouped to satisfy the principles of parsimony. Proteins sharing significant peptide evidence were grouped into clusters. Normalization was performed iteratively (across samples) on intensities. Medians were used for averaging. Spectra data were log-transformed, and weighted by an adaptive intensity weighting algorithm. Differentially expressed proteins were determined by applying a Mann-Whitney U test with significant level of p<0.05 corrected by Benjamini-Hochberg. A volcano plot was generated using the data. Pathway enrichment analysis on differentially expressed proteins (p<0.05) was performed using enricher algorithm to identify overrepresented pathways in HNSCC EVs in GO molecular function, GO biological process, KEGG, and REACTOME.

Western blot. EV lysate or whole cell lysate were prepared on ice using RIPA lysis buffer solution (Thermo Fisher Scientific, Fremont, CA, USA), 1% protease, and phosphatase inhibitor cocktail. Protein concentrations were determined with a Broadford Protein Assay (Thermo Fisher Scientific, Fremont, CA, USA). Lysate protein was subjected to 10% SDS-PAGE and transferred to a nitrocellulose membrane. After blotting, membranes were probed with primary antibody at 4° C. overnight. The following primary Abs were used: anti-CD63 (10628D, Invitrogen, Invitrogen, Carlsbad, CA, USA), anti-CD81 (MA5-13548, Invitrogen, Carlsbad, CA, USA), anti GRP94 (MA3-016, Invitrogen, Carlsbad, CA, USA). After washing three times with TBST (Tris-buffered saline with 0.1% Tween 20 Detergent), membranes were incubated with HRP-conjugated secondary antibodies for 3h at room temperature. Blots were developed with the clarity max western ECL detection system (Bio-Rad, Hercules, CA, USA) according to the manufacturer's instructions and images were captured from iBrightCL1000.

Measurement of thrombin-antithrombin complex (TAT) generation. The isolated tumor EVs and control EVs (at least 6 per group) were incubated in triplicates with freshly drawn human blood (500 μL). The blood was incubated for 2h at 37° C. and the wells were agitated to avoid red blood cell sedimentation. Afterward, blood was aspirated and centrifuged at 2 000 g for 15 min in order to obtain platelet poor plasma and analyzed in respect to changes in TAT and used as marker for assessment of coagulation activation. The TAT ELISA assay (ab108907, Cambridge, MA, USA) was used for quantification of TAT formation in a spectrophotometer.

Quantification of platelet activation by flow cytometry. Platelet activation was quantified using a BD Accuri C6 (BD Biosciences) on single stained platelet sample. HNSCC tumor EVs and control EVs were incubated in triplicates with platelet rich plasma for 2h at 37° C. Afterward, samples were stained with an anti-CD42a/GP9 PE (48042842, Thermo Fisher, Fremont, CA, USA) and an anti-CD62P APC (304910, Biolegend, San Diego, CA, USA) and were incubated in the dark at 4° C. for 40 min. CD42a/GP9 levels were used to identify the platelets and the CD62P levels were used to evaluate the platelet activation.

Quantification of Clotting time. The thrombogenicity of EVs was analyzed using a whole blood clotting time method as described (25). EVs (25 nM) were added to 100 μL of activated blood in a 48 well plate and sample were incubated in room temperature. The non-trapped red blood cells were lysed. Each well was sampled three time and absorbance was assessed at 540 nm using a 96-well plate reader. The size of the clot was inversely proportional to the absorbance value. Four to five samples were used for each condition at each time point.

Enzyme-linked Immunosorbent Assay (ELISA). To verify the proteomic results, ELISA verification was exercised to test the expression of identified proteins. Levels of SERPINA5 (Invitrogen, Carlsbad, CA, USA), ITGB3 (My BioSource, San Diego, CA, USA), ITGA2B (My BioSource, San Diego, CA, USA), AZGP1 (My BioSource, San Diego, CA, USA), and Clr (Abcam, Cambridge, MA, USA) were quantified suing ELISA following the recommended protocols.

Detection of fibrinolysis proteins. For the measurement of fibrinolysis proteins, a LEGENDplex™ Human Fibrinolysis Panel (5-plex) (BioLegend, San Diego, CA, USA) was used. For assay, plasma samples were diluted 10,000-fold with assay buffer. 25 μl of the diluted plasma samples were transferred in duplicates into the 96-well filter plate, plasma samples of 10 healthy volunteers served as control, and the assay was performed following the manufacturer's instructions. All samples were acquired on a ZE5 Cell analyzer (Bio-Rad, Hercules, CA, USA) and analyzed with LEGENDplex™ Data Analysis software. The protein amount was calculated by standard curve and results represent the concentration expressed in ng/mL.

Statistical analysis. Data are expressed as mean±standard error of mean (SEM). Based on the underlying distribution, non-parametric Mann-Whitney U test or parametric Student's t-test was used. For comparing multiple groups, the Kruskal-Wallis test or one-way analysis of variance (ANOVA) were used to compare between different groups and p values were corrected for multiple comparison at the false rate discovery (FDR) of less than 5%. Validation of EV protein expression identified in this study was performed using the area under the curve (AUC) of receiver operating characteristic (ROC) curves on an independent sample of patients using GraphPad Prism v.7 (GraphPad, USA). P value less than 0.05 was considered statistically significant.

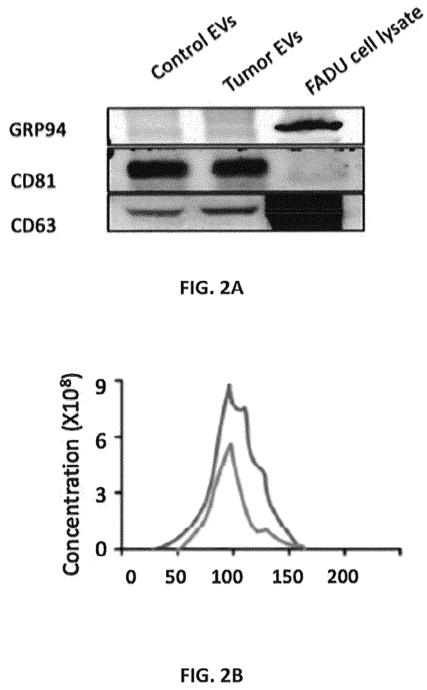

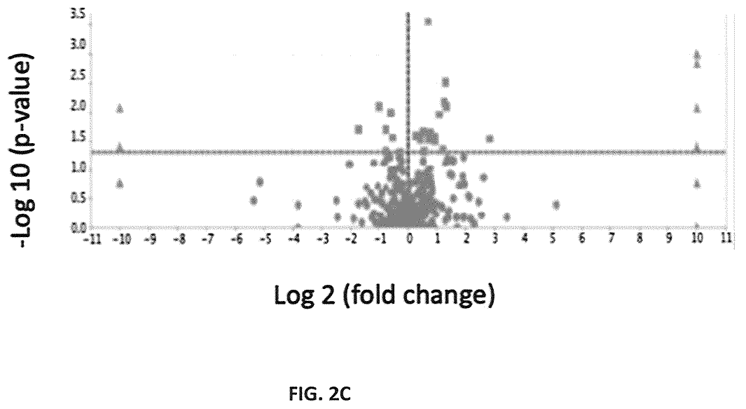

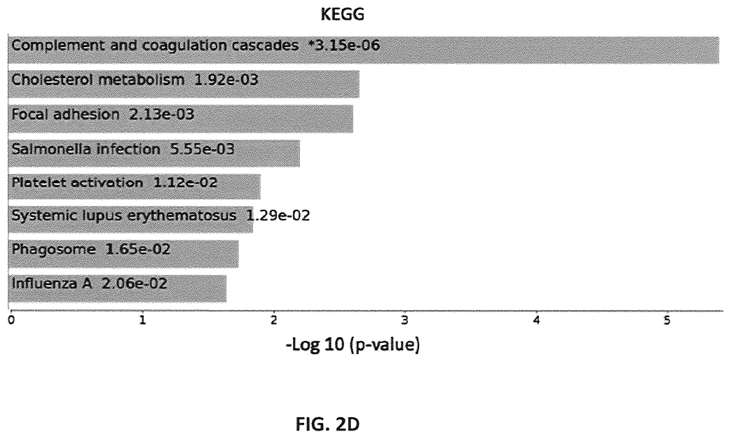

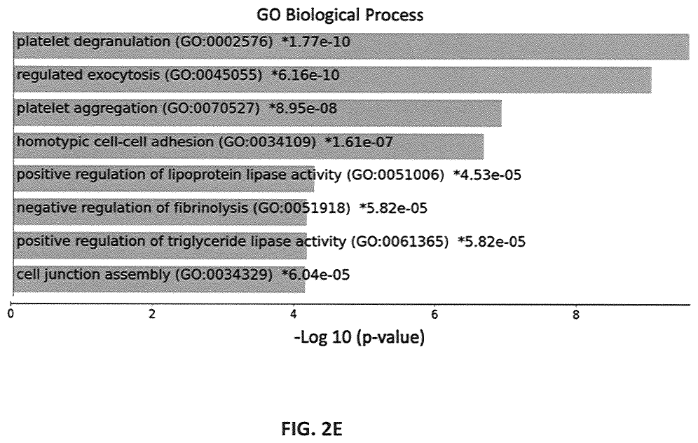

HNSCC-derived EVs carry proteomic signature related to coagulation and hemostasis pathways. EVs isolated from HNSCC patients and healthy controls showed expression of EV-associated proteins, CD63 and CD81 and they were devoid of cellular marker, GRP94 (FIG. 2A). HNSCC EVs showed the mean size of around 100 nm and range of 50 nm-10 nm, whereas healthy EVs showed mean size of 98 nm (FIG. 2B). This size distribution is consistent with the method of isolation to enrich small EV fractions (exosomes). Using label-free proteomic analysis, 258 clusters and 401 proteins were found that were expressed in EVs of HSCC patients and 248 clusters and 392 proteins which expressed in EVs of healthy patients (n=10 control; n=14 HNSCC). Nineteen proteins have been identified that differentially expressed in plasma of HNSCC cancer patients (p≤0.05 after FDR adjustment). Of those 19 proteins, 14 were upregulated and five were downregulated as determined by fold change. The upregulated proteins included Serum albumin (ALB), Alpha-1B-glycoprotein (A1BG), Antithrombin-III (SERPINC1), Apolipoprotein-A-IV (APO-A4), Hemoglobin subunit beta cluster (HBB), Afamin (AFM), Talin-1 cluster (TLN1), Coagulation factor V (F5), Plasma serine protease inhibitor (SERPINA5), Zinc-alpha-2-glycoprotein (AZGP1), Serpin B3 (SERPINB3), Serpin B4 (SERPINB4), and Ig kappa chain V-III region IARC/BL4. Both Serpin B3 and Serpin B4 are squamous-tumor specific antigens and has been suggested as tumor biomarkers for HNSCC, cervical squamous cell cancer, and bladder cancer.

The downregulated proteins included Complement Clr subcomponent (C1r), Thrombospondin-1 (TSP1), Serum amyloid A-1 (SAA1), Immunoglobulin lambda-like polypeptide 1 (IGGLL1), and Serotransferrin (TF). Volcano plot of differentially expressed proteins between HNSCC EVs and healthy EVs based on the fold change is presented at FIG. 2C. Validation experiment in EVs isolated from plasma of an independent cohort of patients showed an increase in AZGP1 and SERPINA5 and a decrease in Clr as predicted by proteomic analysis. Gene ontology and pathway analysis was performed to identify functional pathways associated with the differentially detected proteins. Examining the protein with high concentration in HNSCC EVs revealed dysregulation of pathways related to complement and coagulation cascades (KEGG), platelet degranulation (GO Biological process), regulated exocytosis (GO Biological process), and platelet aggregation (GO Biological process) (FIG. 2D, E).

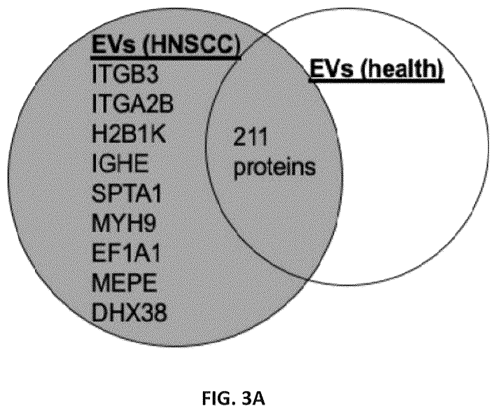

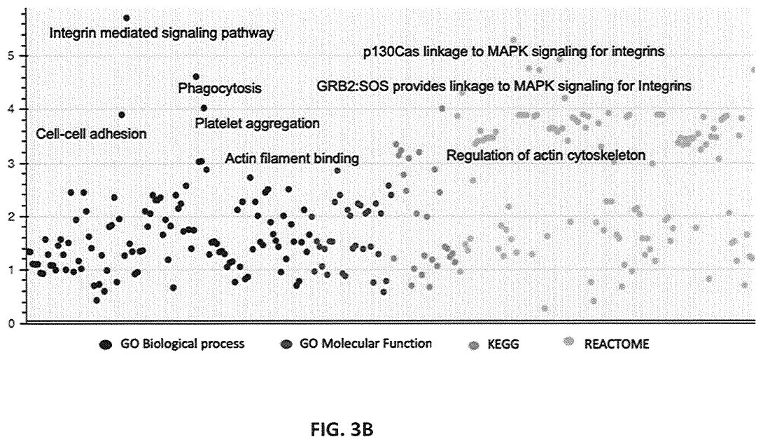

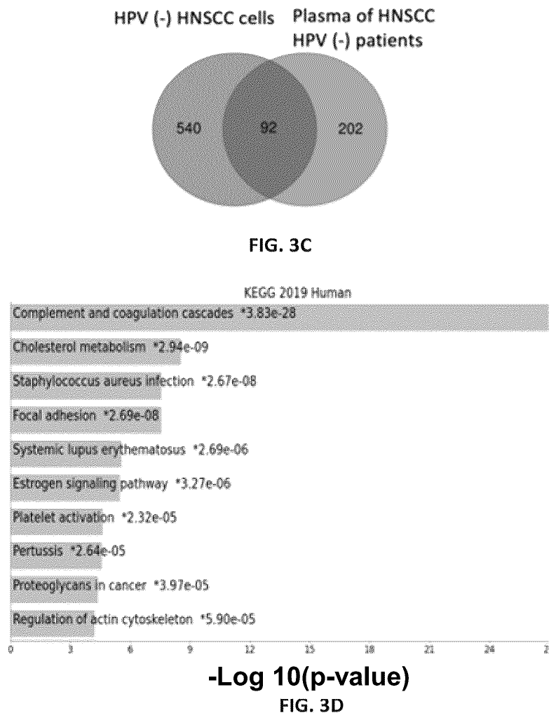

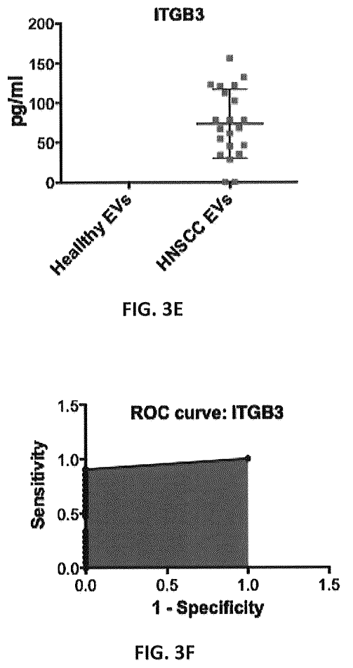

HNSCC EVs carry several unique proteins that can be used for biomarker discovery of HNSCC. To identify the HNSCC specific EV-associated biomarkers, proteins were identified that were present in HNSCC EVs but not in healthy EVs. Those proteins included Integrin beta-3 (ITGB3), Integrin alpha-IIb (ITGA2B), Histone H2B type 1-K (H2B1K), Ig epsilon chain C (IGHE), Spectrin alpha chain, erythrocytic 1 (SPTA1), Myosin-9 (MYH9), Elongation factor 1-alpha 1 (EF1A1), Matrix extracellular phosphoglycoprotein (MEPE), Pre-mRNA-splicing factor ATP-dependent RNA helicase PRP16 (DHX38) (FIG. 3A)). These proteins were associated with integrin mediated signaling pathway, platelet aggregation, regulation of cytoskeleton, phagocytosis, cell-cell adhesion, actin filament binding, regulation of actin cytoskeleton, and p130cas linkage to MAPK signaling for integrins (FIG. 3B). The proteomic profile of EVs isolated from plasma of patients with HNSCC were next compared with HPV negative HNSCC cell lines. 92 proteins were found that overlapped between HNSCC EVs and HNSCC HPV-cell lines (FIG. 3C), indicating that HNSCC tumor is the likely origin of those proteins. Consistent with what was found in the differential expression analysis of plasma HNSCC EVs versus healthy EVs, and in HNSCC-specific pathway enrichment analysis, complement and coagulation cascades, regulation of actin cytoskeleton, and cholesterol metabolism were among the most enriched pathways (FIG. 3D). In an independent cohort of patients, circulating ITGB3 was detected expressing EVs in 90% of HNSCC patients, where this protein was absent in EVs isolated from plasma of healthy patients (FIG. 3E). Construction of ROC curve showed high accuracy of this marker for detection of HNSCC (area under the curve (95% confidence interval):0.93:0.84-1; p<0.0001) (FIG. 3F).

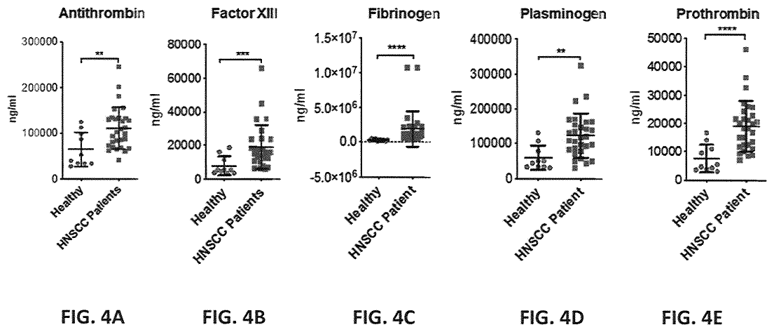

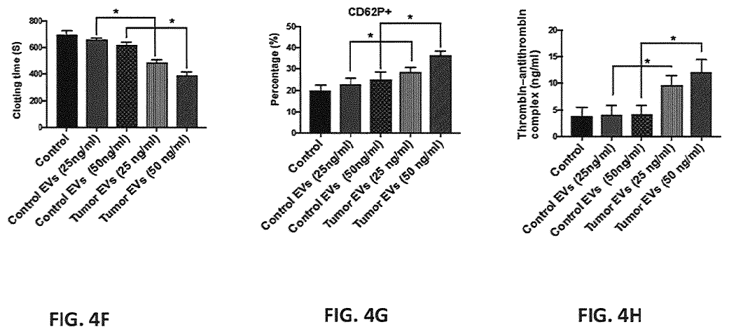

Human thrombosis markers are increased in HNSCC patients and HNSCC tumor-derived EVs induce coagulation and platelet degradation. As coagulation cascades and platelet activation were among the most statistically significant enriched pathways in both EVs isolated from HNSCC cell lines and plasma of HNSCC patients, human thrombosis status of HNSCC patients was analyzed next. Results showed that antithrombin, factor XIII, fibrinogen, plasminogen, and prothrombin were significantly increased in plasma of HNSCC patients compared to the controls (p<0.005) (FIG. 4A-E). Coculture of tumor EVs with whole blood significantly increased the formation of thrombin-antithrombin complex in a dose dependent manner compared to the control EVs (FIG. 4F). Moreover, tumor EVs induced an increase in the activation of platelets as measured by percentage of CD62P+ cells in the whole platelet population (CD42a/GP9) (p<0.05) (FIG. 4G). HNSCC tumor derived EVs significantly reduced clotting time in a dose dependent manner (p<0.05) (FIG. 4H).

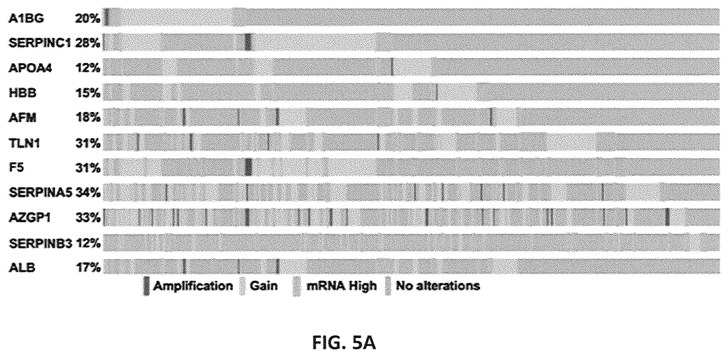

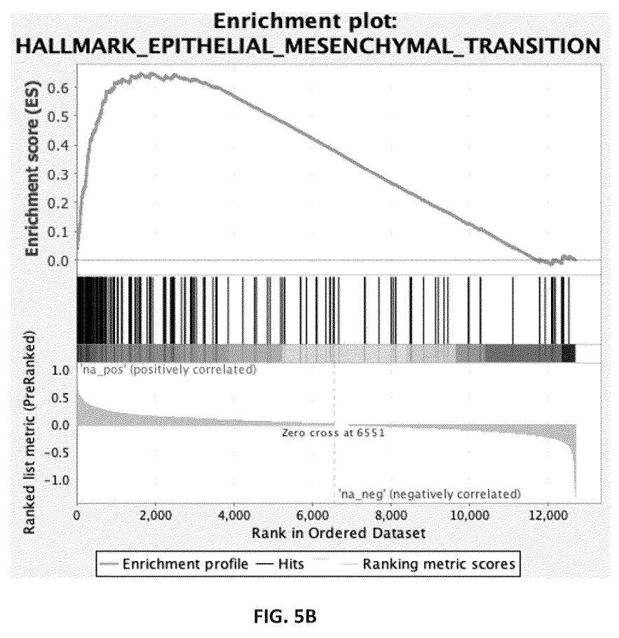

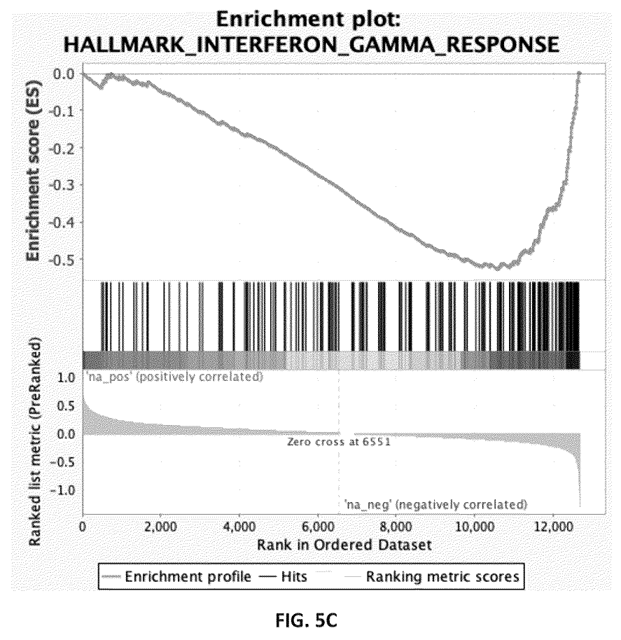

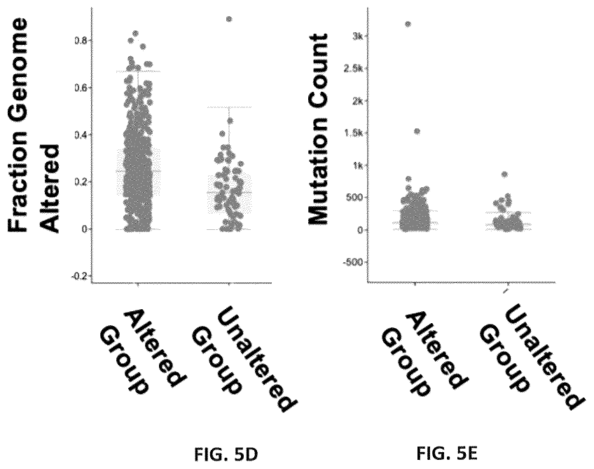

Hemostasis related genes increased in HNSCC EVs are genomically altered in HNSCC and pan-cancer tumors. Bioinformatic analysis on TCGA data on overexpressed coagulation and hemostasis markers in HNSCC EVs (A1BG, SERPINC1, APOA4, HBB, AFM, TLN1, F5, SERPINA5, AZGP1, SERPINB3, ALB), showed presence of amplifications, gains, and high mRNA levels in those transcripts in HNSCC (FIG. 5A). More than 30% of samples for each of the following proteins SERPINA5, F5, TLN1, and AZGP1 demonstrated genomic alterations in the form of gain, amplification, and high mRNA levels. 28% and 20% of the samples for A1BG and SERPINC1 showed these same changes, respectively. These results show the same pattern of increase in the tumor of HNSCC patients and EVs. Interestingly, patients with high gain, amplification, and high mRNA of the upregulated genes showed significant co-occurrence of those genes (Table 1).

TABLE 1
Probability of co-occurrence of genomic alteration of highly
abundant hemostasis-associated genes in HNSCC EVs.

| | | Log 2 Odds | | | |
|---|---|---|---|---|---|
| A | B | Ratio | p-value | q-value | Tendency |
| AFM | ALB | >3 | <0.001 | <0.001 | Co-occurence |
| SERPINC1 | F5 | >3 | <0.001 | <0.001 | Co-occurence |
| SERPINC1 | AZGP1 | 1.686 | <0.001 | <0.001 | Co-occurence |
| F5 | AZGP1 | 1.459 | <0.001 | <0.001 | Co-occurence |
| APOA4 | HBB | 2.306 | <0.001 | <0.001 | Co-occurence |
| SERPINC1 | AFM | 1.663 | <0.001 | <0.001 | Co-occurence |
| SERPINC1 | ALB | 1.592 | <0.001 | <0.001 | Co-occurence |
| A1BG | SERPINA5 | 1.288 | <0.001 | <0.001 | Co-occurence |
| AZGP1 | ALB | 1.216 | <0.001 | 0.003 | Co-occurence |
| SERPINA5 | AZGP1 | 0.936 | <0.001 | 0.004 | Co-occurence |
| HBB | SERPINA5 | 1.085 | 0.003 | 0.010 | Co-occurence |
| AFM | AZGP1 | 0.946 | 0.004 | 0.016 | Co-occurence |
| AFM | F5 | 0.868 | 0.009 | 0.03 | Co-occurence |
| A1BG | F5 | 0.824 | 0.010 | 0.03 | Co-occurence |
| F5 | ALB | 0.868 | 0.011 | 0.03 | Co-occurence |
| A1BG | AZGP1 | 0.806 | 0.011 | 0.03 | Co-occurence |
| A1BG | SERPINC1 | 0.797 | 0.015 | 0.038 | Co-occurence |

*q-value: corrected for multiple correlation based on false discovery rate.

There were many genes found to have high expression changes that are in association with one another. High levels of SERPINC1 were associated with high levels of F5, AZGP1, ALB, AFM, and SERPINA5. Patients with high AFM levels also had high ABM, AZGP1, and F5 levels. High levels of A1BG were found to be associated with high levels of AERPINA5, F5, AZGP1, and SERPINC1. We also found that samples which had gain, amplification and high mRNA of A1BG, SERPINC1, APOA4, HBB, AFM, TLN1, F5, SERPINA5, AZGP1, SERPINB3, ALB were associated with tumors with higher fraction of genomic changes and higher mutation burden (p<0.05) (FIG. 5B, C).

EVs signature was associated with positive enrichment of epithelial-mesenchymal transition and negative enrichment of interferon gamma signaling pathways. GSEA analysis on transcripts which were significantly associated with high levels of the altered proteins were found to have a positive enrichment score and upregulation in EMT and a negative enrichment score and downregulation of the interferon gamma response pathway (FIG. 5D, E). Activation of EMT has been found to be associated with an increase in tumor invasion and metastasis. Enhanced interferon gamma response induces immune system activation and eliminates the cancer cells, so a decrease in activation in this pathway will result in an immunosuppressive profile. Other associated positively enriched pathways were myogenesis, angiogenesis, coagulation, MYC targets, reactive oxygen species, and hedgehog signaling. Among other negatively enriched pathways include Interferon alpha response, early and late estrogen response, allograft rejection, G2M Checkpoint, heme metabolism, protein secretion and inflammatory response.

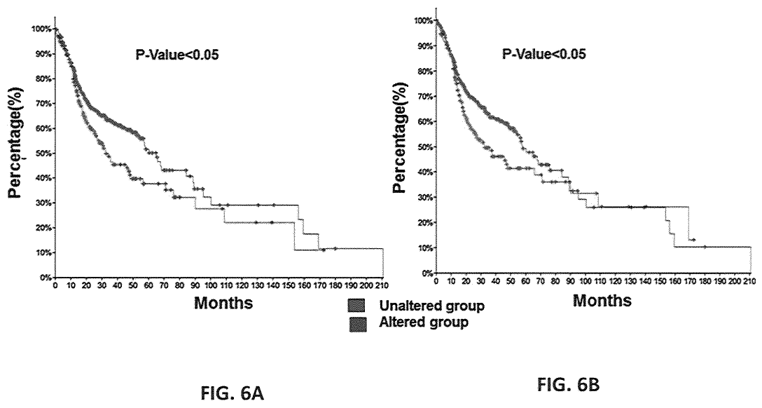

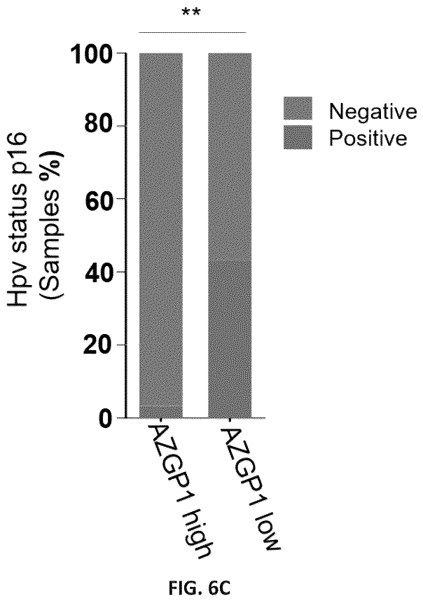

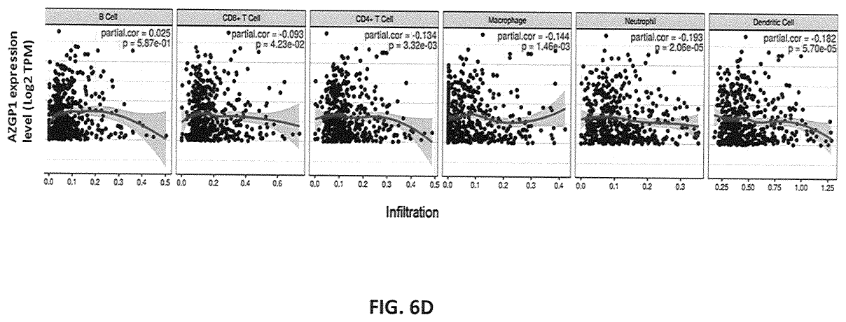

Increase in HNSCC EV associated proteins are associated with lower survival in HNSCC and an immunosuppressive TME. We also assessed the effect of genomic alterations in EV-associated proteins on survival; patients with AZGP1 amplification, gain and mRNA expression had a lower overall survival compared to the unaltered group (p<0.05) (FIG. 6A). Patients with the same high expression changes in SERPINA5 had significantly lower survival compared to patients with unaltered expression of SERPINA5 (p<0.05) (FIG. 6B). Interestingly, higher levels of AZGP1 was associated with HPV-cases in HNSCC TCGA cohort (FIG. 6C). In fact, an increase in AZGP1 expression was found to be associated with a lower abundance of CD4+ and CD8+ T cells, macrophages, neutrophils, and dendritic cells in HNSCC, indicating presence of more immune quiet and immune suppressive TME (FIG. 6D).

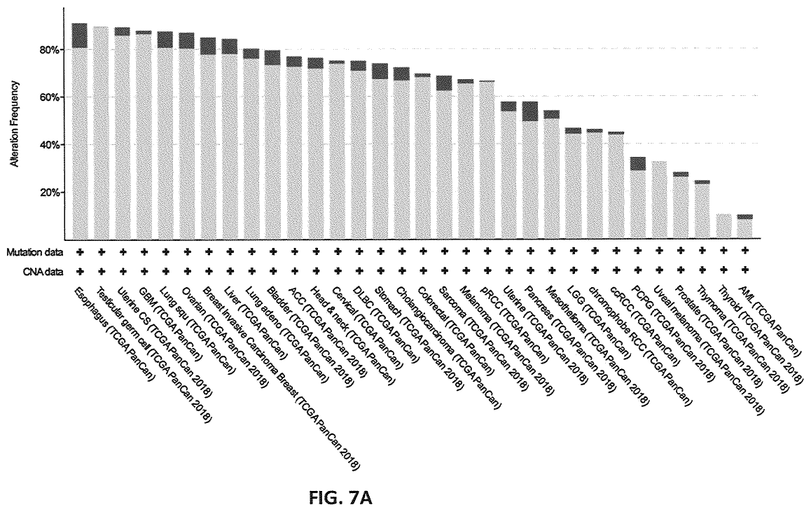

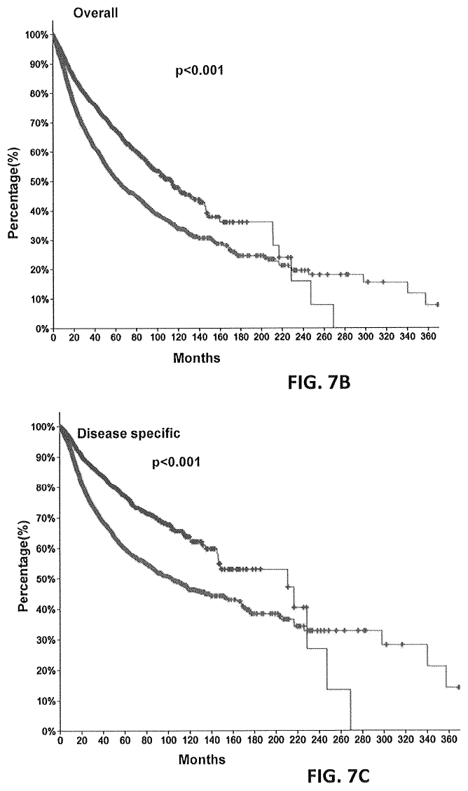

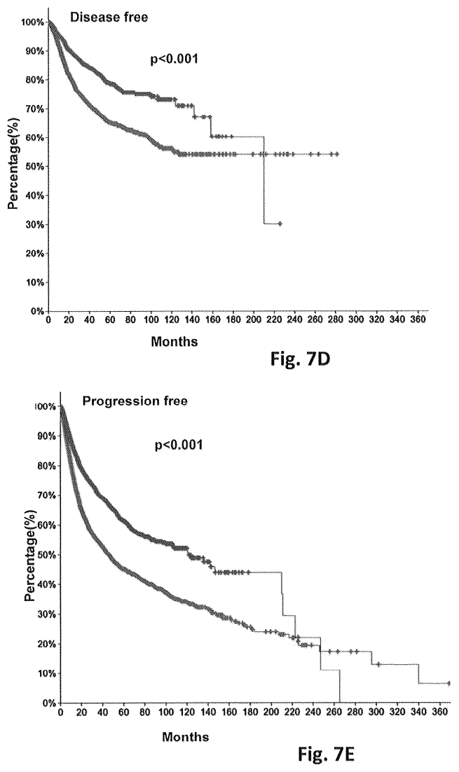

Genomic alterations and increase EV associated proteins were associated with lower survival in HNSCC and an immunosuppressive TME. The effect of EV-enriched altered upregulated proteins was again assessed using the pan-cancer TCGA data to see their expression in other cancers. We found similar genomic alterations of EV-enriched proteins in other solid tumors such as bladder and lung cancer (FIG. 7A). Overall survival (FIG. 7B), the disease-free survival (FIG. 7C), disease-specific survival (FIG. 7D), and progression-free survival (FIG. 7E) were all lower in the group of patients with altered upregulated proteins.

### EXAMPLE

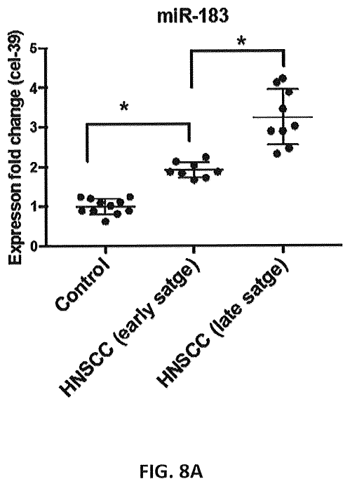

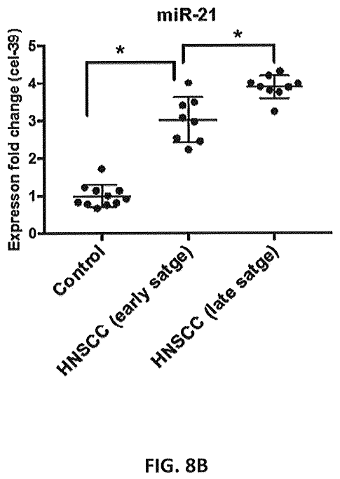

To demonstrate the proof of concept that HNSCC specific EVs/exosomes can be used for detection of cancer and by enriched isolation of cancer EVs (that can be achieved via immune magnetic beads or aptamer), we validated our finding in 28 individuals with HNSCC and age- and gender matched control. The results showed that isolation of EVs/exosomes based on expression of surface ITGB3 expression can lead to high specificity and sensitivity for detection of cancer, and the levels of miR-21 (FIG. 8B) and miR-183 (FIG. 8A) packaged into the ITGB3 expressing exosomes/EVs which are specific to HNSCC can be used to identify patients with HNSCC and discriminate early stage disease from late stage of HNSCC.

Exosomes/EVs were isolated by ITGB3-specific immune magnetic beads isolation. RNA from exosomes/EVs were isolated by an RNA isolation kit (Zymo) and the levels of miR-21 and miR-183 were quantified using a TaqMan miR assay. The miRs were normalized to cel-39. The data is presented as fold change compared to the control. (* indicated p-value less than 0.05.) FIG. 8A illustrates fold change of expression of miR-183 for late stage HNSCC versus fold change for early stage HNSCC and controls. FIG. 8B illustrates fold change in expression of miR-21 for late stage HNSCC and fold change for early stage HNSCC versus control. Early stage disease has more than 90% survival and late stage has around 20-30% survival so that can affect the treatment outcome, patient survival, and treatment cost.

In this study, we found proteomic changes in HNSCC EVs associated with dysregulation of coagulation, hemostasis, and platelet degranulation pathways. Our mechanistic studies confirmed the role of EVs in inducing coagulation and platelet activation. We showed that tumor EVs but not healthy EVs could induce thrombin-antithrombin complexes and activate platelets. We also found proteins that were exclusively expressed in HNSCC EVs, including integrin proteins ITGB3 and ITGA2B, which are plasma membrane proteins and can be used for tumor-specific EV isolation and biomarker discovery. Interestingly, both proteins were present in EVs produced by HPV negative HNSCC cell lines, indicating these proteins' tumor cell origin. ITGB3 showed high accuracy for the diagnosis of HNSCC. We also found a high abundance of squamous tumor antigens, Serpin B3 and Serpin B4, in EVs isolated from HNSCC patients' plasma. These data could be leveraged in future studies to produce biomarkers for HNSCC.

Interestingly, our studies on tumor genomic alteration in HNSCC and pan-cancer studies showed that proteins which are increased in EVs are altered in HNSCC and other solid tumors in the form of genomic amplification, gain, and high mRNA. In particular, four genes in tumor EVs, SERPINA5, AZGP1, F5, and TLN1, had prevalent alterations (>30%), identified by increased amplification, gain, or high mRNA. We also found significant co-occurrence of those genes in the tumor tissue and their association with tumor survival. While all genomic alterations in the form of amplification, gain and high mRNA were observed, an increase in copy number was the most common genomic alteration of dysregulated genes. Patients with genomic changes reflected in their EVs had a higher fraction of the genome altered and mutation count. Interestingly, the top abundant four genes had a significant co-occurrence, indicating that high expression of SERPINA5, AZGP1, F5 and TLN1 all happen in the same patient population. When looking at survival associated with the particular coagulation signature of EVs, our data suggested that a higher fold change in AZGP1 was associated with lower survival. Interestingly, those genomic changes were common in other solid tumors and were associated with pan-cancer poor overall survival, disease-free survival, and progression-free survival.

The coagulation cascade is a complex system involving enzymatic activation of pro-cofactors and proteins that polymerize fibrin and stimulate platelets to prevent blood loss (30). In homeostasis, coagulation is a balance between bleeding and clotting. In TME, it has been shown that pro-coagulation factors, such as tissue factor (TF) expression, are upregulated in the environment and in tumor cells and are overexpressed in carcinomas and other neoplasia. This expression in tumor cells has been linked to mutant oncogenes, such as the human epidermal growth factor receptor 2 (HER-2) and EGFRvIII, as well as loss of tumor suppressor genes, such as p53 and PTEN. It has also been shown that a higher level of exposed TF on the surface of tumor cells is related to hypercoagulability. Procoagulant and fibrinolytic proteins are released by cancer cells and bind to blood cells, causing prothrombotic events that would be induced in normal cells. It has been suggested that activation of coagulation pathways and platelet aggregation induce immune evasion in TME and promote metastasis and invasion.

Thrombosis is one of the leading causes of death associated with cancer and is thought to be associated with the hypercoagulable state induced from oncogene activation and the TME. Many studies have shown a disruption of the coagulation cascade among various cancers due to findings that thromboembolic incidences are correlated to cancer prognosis. Thrombosis due to activation of coagulation factors also negatively affects survival. For instance, in non-small cell lung carcinoma, patients with elevated fibrinogen levels and prolonged prothrombin time and international normalized ratio had lower overall survival over 60 months compared to patients without cancer. In addition, there is a higher risk of invasion and aggressive metastasis in tumors that activate the coagulation cascade. Abnormal coagulation in malignant tumors is thought to be related to the inflammatory reactions caused by the cancer. Hypercoagulable state and its association with tumor progression have been reported in lung cancer, pancreatic cancer, and gastrointestinal cancer. For instance, factor VIII was also greatly elevated in advanced-stage lung cancer compared to stage I, II, and III patients. In breast cancer, abnormal activation of coagulation pathways was associated with metastasis and activation of the NF-kB. Our novel data, for the first-time, demonstrated increase in coagulation markers in plasma of patients with HNSCC. Specifically, antithrombin, factor XIII, fibrinogen, plasminogen, and prothrombin were significantly increased in plasma of HNSCC patients compared to the controls. Mechanistically, HNSCC EVs induced coagulation, decreased clotting time, and induced platelet activation. Role of EVs from other sold tumors should be further assessed in mechanistic studies.

Few studies evaluated role of circulating EVs in thrombosis and thromboembolisms away from the site of the primary tumor in cancer patients. In ovarian cancer, it has been shown that damage to the tumor vasculature allows coagulation factors to enter the tumor and form surface TF/FVIIa/FXa complexes. Cancer cells that release these complexes can transport them downstream to have procoagulant effects and contribute to regulation of the immune system. Elevation of EVs with TF in cancer patients has been associated with an increased risk of thrombosis because TF bound to EVs on the surface act as potent initiators of the coagulation cascade. Studies showed that there is an in increase in EV-TF expression and moderate association with d-dimer levels in advanced colorectal cancer, glioblastoma, and prostate cancer, increasing risk for thrombosis. In pancreatic ductal adenocarcinoma, there is a correlation between EV-TF and venous thromboembolism, as patients with a high expression of TF on circulating neoplastic cells had five times the thromboembolism rate as patients with low TF expression. It was also concluded that a high incidence of venous thromboembolism and TF expression in vivo is associated with ovarian clear-cell carcinoma. The high expression of TF and coagulation complexes in exosomes from cancer patients is thought to be correlated with increased thrombotic incidence and is being considered as a biomarker for cancer progression.

In this study, HNSCC patients with hemostasis-related genomic alterations showed positive enrichment of EMT pathways compared to the group without those changes. In breast cancer, there is a higher expression of TF expressed on EMT positive cells and those EMT positive cells promote coagulant activity. We also found decrease in the interferon gamma response of tumors with genomic alterations in hemostasis genes. Interferon gamma signaling is one of the important determinants of response to the immunotherapy in patients and downregulation of this response may indicate lower response to immunotherapy. These correlations should be evaluated in future studies. In this study, we found a negative association between high levels of AZGP1 and survival in HNSCC. Although the role of AZGP1 is still relatively unknown, AZGP1 is a soluble glycoprotein whose expression is regulated by histone acetylation and it has implications in lipid mobilization and cachexia associated with the malignancy. AZGP1 expression is also thought to play a role in an anti-tumor immune response due to its structural similarity to the major histocompatibility complexes. Interestingly, our data indicates the higher levels of AZGP1 are associated with HPV negative tumors and AZGP1 expression was found to be associated with a lower expression of CD4+ and CD8+ T cells, macrophages, neutrophils, and dendritic cells in HNSCC, indicating presence of more immune-suppressive TME. These findings indicate the potential of AZGP1 as possible disease biomarker in HPV negative HNSCC.

The results indicate that specific proteomic changes such as ITGB3, ITGA2B can be detected exclusively in HNSCCC EVs and could serve as HNSCC biomarkers. HNSCC EVs were enriched in proteins related to coagulation and hemostasis and functionally induced activation of coagulation pathways and platelet activation. Proteomic changes in EVs correlated with tumor-specific genomic alteration in hemostasis-related genes. Changes in hemostasis-related genes were associated with patient survival and patients with high levels of those transcript demonstrated activation of pathways related to immunosuppressive TME. These data underline the possibility of use of EVs as platform for precision medicine and biomarker discovery as disease-associated biomarkers.

### Definitions

As used herein, a “subject” or “patient” can be any mammal, e.g., a human, a primate, mouse, rat, dog, cat, cow, horse, pig, sheep, goat, camel. In a preferred aspect, the subject is a human. A subject can be diagnosed with cancer.

The sample can be a biological sample. As will be appreciated by those in the art, the sample may comprise any number of things, including, but not limited to: cells (including both primary cells and cultured cell lines) and tissues (including cultured or explanted). In aspects, a tissue sample (fixed or unfixed) is embedded, serially sectioned, and immobilized onto a microscope slide. As is well known, a pair of serial sections will include at least one cell that is present in both serial sections. Structures and cell types, located on a first serial section will have a similar location on an adjacent serial section. The sample can be cultured cells or dissociated cells (fixed or unfixed) that have been immobilized onto a slide.

The sample can be obtained from virtually any organism including multicellular organisms, e.g., of the plant, fungus, and animal kingdoms; preferably, the sample is obtained from an animal, e.g., a mammal. Human samples are particularly preferred.

In some aspects, the preceding methods are used in the clinical assessment of a subject. As used herein the term “clinical assessment of a subject” can comprise producing a report that predicts or diagnoses a condition in a subject, determine a subject's predisposition to a condition, monitors the treatment of a condition in a subject, diagnoses a therapeutic response of a disease in a subject and prognose the disease, disease progression, or response to particular treatment of a disease in a subject.

The terms “cancer” and “cancerous” refer to or describe the physiological condition in mammals that is typically characterized by unregulated cell growth. Included in this definition are benign and malignant cancers. Examples of cancer include but are not limited to, carcinoma, lymphoma, blastoma, sarcoma, and leukemia. More particular examples of such cancers include adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma. Other examples include breast cancer, lung cancer, lymphoma, melanoma, liver cancer, colorectal cancer, ovarian cancer, bladder cancer, renal cancer or gastric cancer. Further examples of cancer include neuroendocrine cancer, non-small cell lung cancer (NSCLC), small cell lung cancer, thyroid cancer, endometrial cancer, biliary cancer, esophageal cancer, anal cancer, salivary, cancer, vulvar cancer or cervical cancer.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

The terms “response or “benefit” is used in the broadest sense and refers to any desirable effect and specifically includes clinical benefit as defined herein. Clinical benefit can be measured by assessing various endpoints, e.g., inhibition, to some extent, of disease progression, including slowing down and complete arrest; reduction in the number of disease episodes and/or symptoms; reduction in lesion size; inhibition (i.e., reduction, slowing down or complete stopping) of disease cell infiltration into adjacent peripheral organs and/or tissues; inhibition (i.e. reduction, slowing down or complete stopping) of disease spread; decrease of auto-immune response, which may, but does not have to, result in the regression or ablation of the disease lesion; relief, to some extent, of one or more symptoms associated with the disorder; increase in the length of disease-free presentation following treatment, e.g., progression-free survival; increased overall survival; higher response rate; and/or decreased mortality at a given point of time following treatment.

The term “anti-cancer therapy” is used in the broadest sense and refers to any method known in the art for the treatment of cancer. Anti-cancer therapy can include, but is not limited to, the administration of chemotherapeutic agents, the administration of anti-cancer agents, radiation treatment, immunotherapy, surgery, radiation therapy, targeted therapy, hormone therapy and stem cell transplant. Anti-cancer therapy can comprise administering to the subject a therapeutically effective dose of at least one class of drugs. The terms “effective amount” and “therapeutically effective amount” of a drug, agent or compound of the invention is meant a nontoxic but sufficient amount of the drug, agent or compound to provide the desired effect, for example, a response or benefit in the subject.

Classes of anti-cancer agents can include, but are not limited to, antibodies.

The methods of the present disclosure can use reference genes or proteins to normalize the measured abundance of other genes and biomarkers. Normalization can be used to control for experimental variation to facilitate more accurate comparisons between measurements from different samples. In a non-limiting example, a first sample from a first patient and a second sample from a second patient are analyzed. The first sample from the first patient may be more concentrated than the second sample from the second patient, meaning more nucleic acids are extracted from the first sample than are extracted from the second sample. Thus, if the expression level of a particular gene is measured in the two samples, the expression level of the gene will appear higher in the first sample than the second sample, simply because there are more nucleic acid molecule in the first sample. To more accurately compare between the two samples, the two measured expression levels can be normalized.

Normalization can also be used to control for unwanted biological variation. In a non-limiting example, biological variation can result from some feature of the patient or the sample collection that is not relevant to the methods of the present disclosure, such as blood-exosome concentration due to high or low blood pressure, variations created by collecting samples at different times of the day and variation to due patient age or patient sex.

In methods of the present disclosure, the measured expression level of a particular gene can be normalized using methods known in the art. In a non-limiting example, normalization can be achieved by dividing the measured expression level of a gene of interest by a reference gene. Useful reference genes are genes that show a low variation in their expression level across a variety of different samples and patients. For example, a useful reference gene will show the same expression level in samples derived from subjects who have cancer and in samples derived from subjects who do not have cancer. In another example, a useful reference gene will show the same expression level in samples derived from a subject with cancer before treatment with an anticancer therapy and in in samples derived from a subject with cancer after treatment with an anti-cancer therapy. The variation in expression level can be quantified by different methods known in the art. For example, the variation in expression level of a gene can be quantified by calculating the coefficient of variation in the expression level of a particular gene across a set of different samples.

While the disclosed subject matter is described herein in terms of certain non-limiting exemplary embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments. In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of non-limiting example embodiments of the disclosed subject matter has been presented for purposes of illustration and description. It is not intended to be exhaustive or to limit the disclosed subject matter to those embodiments disclosed herein

### LIST OF ABBREVIATIONS

A1BG: Alpha-1B-glycoprotein

AFM: Afamin

ALB: Serum Albumin cluster

APO-A4: Apolipoprotein-A-IV

AZGP1: Zinc-alpha-2-glycoprotein

Clr: Complement component 1r

EGFRvIII: Epidermal growth factor receptor III

ELISA: Enzyme-linked Immunosorbent Assay

EMT: Epithelial-mesenchymal transition

EVs: Extracellular vesicles

F5: Coagulation factor V

FDR: False discovery rate

FVIIa: Activated blood coagulation factor VII

FXa: Activated blood coagulation factor X

GSEA: Gene set enrichment analysis

HBB: Hemoglobin subunit beta cluster

HER-2: Human epidermal growth factor receptor 2

HNSCC: Head and neck squamous cell carcinomas

HPV: Human papilloma virus

MP: Microparticles

NF-kB: Nuclear Factor kappa-light-chain-enhancer of activated B cells

NF-kB: Nuclear Factor kappa-light-chain-enhancer of activated B cells

NSCLC: Non-small cell lung carcinoma

NTA: Nanoparticle Tracking Analysis

PTEN: Phosphatase and TENsin homolog

SD: Standard deviation

SEM: mean±standard error of mean

SERPINA5: Plasma serine protease inhibitor

SERPINB3: Serpin B3

SERPINB3: Serpin B3 cluster

SERPINB4: Serpin B4

SERPINC1: Antithrombin-III

TCGA: Cancer genome atlas

TF: Tissue factor

TLN1: Talin-1 cluster

TME: Tumor microenvironment

VTE: Venous thromboembolism

What is claimed is:

1. A method of treating a squamous cancer in a subject comprising identifying, or having identified, the presence or absence of squamous cancer in a subject comprising determining the expression level of at least one protein selected from ITGB3, ITGA2B, H2B1K, IGHE, SPTA1, MYH9, EF1A1, MEPE, DHX38, and combinations thereof in a biological sample from the subject, and, wherein a squamous cancer in a subject is identified as present, treating the squamous cancer with surgery, radiation therapy, chemotherapy, intravesical therapy, anti-cancer therapy, immunotherapy, intravesical immunotherapy, targeted-drug therapy, intravesical chemotherapy or any combination thereof.

2. The method of claim 1, wherein the identifying, or having identified, the presence or absence of squamous cancer in a subject comprises:
determining the expression level of at least one protein selected from ITGB3, ITGA2B, H2B1K, IGHE, SPTA1, MYH9, EF1A1, MEPE, DHX38, and combinations thereof in a biological sample from the subject;
determining at least one reference value in the biological sample;
normalizing the expression level of the at least one protein to the at least one reference value to obtain a normalized expression level of the at least one protein;
comparing the normalized expression level of the at least one protein to a predetermined cutoff value; and
identifying the presence of squamous cancer in the subject when the normalized expression level of the at least one protein is greater than the predetermined cutoff value or identifying the absence of squamous cancer in the subject when the normalized expression level of the at least one protein is less than or equal to the predetermined cutoff value.

3. The method of claim 1, wherein the identifying, or having identified, the presence or absence of squamous cancer in a subject comprises:
determining the expression level of at least one protein selected from ITGB3, ITGA2B, H2B1K, IGHE, SPTA1, MYH9, EF1A1, MEPE, DHX38, and combinations thereof in a biological sample from the subject;
determining at least one reference value in the biological sample;
normalizing the expression level of the at least one protein to the at least one reference value to obtain a normalized expression level of the at least one protein;
comparing the normalized expression level of the at least one protein to a predetermined cutoff value; and wherein the biological sample comprises at least one extracellular vesicle (EV) nucleic acid extracted from at least one EV; identifying the presence of squamous cancer in the subject when the normalized expression level of the at least one protein is greater than the predetermined cutoff value or identifying the absence of squamous cancer in the subject when the normalized expression level of the at least one protein is less than or equal to the predetermined cutoff value.

4. The method of claim 1, wherein the squamous cancer is head and neck cancer.

5. The method of claim 3, wherein the at least one EV nucleic acid comprises EV DNA, EV RNA or a combination of EV DNA and EV RNA.

6. The method of claim 5, wherein the at least one EV nucleic acid comprises EV RNA.

7. The method of claim 3, wherein the at least one EV is isolated from a bodily fluid sample from the subject.

8. The method of claim 3, wherein the bodily fluid sample is filtered or pre-processed to remove cells, cellular debris or any combination thereof prior to the isolation of at least one EV.

9. The method of claim 8, wherein the at least one EV is isolated from the bodily fluid sample by a method comprising filtration, size exclusion chromatography, ion exchange chromatography, density gradient centrifugation, centrifugation, differential centrifugation, immunoabsorbent capture, affinity purification, affinity enrichment, affinity exclusion microfluidic separation, ultracentrifugation, nanomembrane ultrafiltration or any combination thereof.

10. The method of claim 2, wherein the predetermined cutoff value has a sensitivity of at least 50%.

11. The method of claim 2, wherein the predetermined cutoff value has a specificity of at least 50%.

12. The method of claim 1, wherein the surgery comprises resection of the tumor.
